(19) United States
(12) Patent Application Publication
(10) Pub. No.: US 2004/0092802 A1
(43) Pub. Date: May 13, 2004

(54) Epithelial diagnostic aid

(76) Inventors: Michael Roger Cane, Royston (GB); Symon D?apos;oyly Cotton, Great Gransden (GB); Matthew Alexander Schumann, Bourn (GB); Michael Andrew Beadman, Nr Royston (GB); Thomas Scott Carter, Trumpington (GB); Philip James Churchill White, St Albans (GB); Rebeca Lapping, Cherry Hinton (GB); Jeremy Carey, Cambridge (GB)

Correspondence Address: Richard M Goldberg, Suite 419, 25 East Salem Street, Hackensack, NJ, 07601, US

(21) Appl. No.: 10/332,222

(22) PCT Filed: Jul. 4, 2001
(86) PCT No.: PCT/GB01/03011

(30) Foreign Application Priority Data
Jul. 7, 2000 (GB) 0016690.0
May 23, 2001 (GB) 0112501.2

(51) Int. Cl. A61B 5/00

(52) U.S. Cl. 600/306

(57) ABSTRACT

The present invention relates to devices and methodology for determining the variation in concentration of chromophores within an epithelial tissue. The devices comprise a means of illuminate the skin at one or more points and at wavelengths corresponding to specific chromophores. The device further comprises a detection means configured to detect the intensity of light remitted from the epithelial surface, a processing means configured to interpret image data sets obtained by the detection means and an output display to display the results or significance of the results. The invention further extends to a method of interpreting the distribution of chromophores within a lesion of an epithelial tissue. The method comprising the steps of illuminating an epithelial surface, determining the intensity of remitted light, analysing said intensity data to determine variation in the intensity across the lesion and providing an output corresponding to the extend of variation across the lesion. One aspec of the invention relates to a hand-held design of the device for implementing the aforementioned method. Another aspect of the invention relates to the design of the patient-contacting part of the device to prevent of contaminants and to improve the diagnostic effectiveness of the device.

### BACKGROUND OF THE INVENTION

1. Field of the Invention

The present invention relates to: i) a diagnostic aid for the examination of lesions of epithelial surfaces; ii) a skin diagnostic aid and more particularly to a nose cone for use with skin illumination and remitted light detection apparatus as described in UK Patent Application Number 00 10 888.6.

2. Background to the Invention

It is known that epithelial surfaces, such as the skin, comprise a variety of chromophores disposed within the constituent layers of the epithelial tissue. In addition, inhomogeneities in the distribution of specific chromophores within the epithelial tissue can be correlated with specific abnormalities (referred to herein as lesions).

In the case of skin, the conventional approach for diagnosing skin ailments involves the examination of the surface characteristics of a lesion. In addition, and dependant on the skin condition, a proportion or entire area of a skin lesion may be surgically excised for histological examination under a microscope.

There are a variety of epithelial tissue conditions where the provision of histological information rapidly would be a valuable adjunct to enable the efficient diagnosis of an ailment. In the example of a malignant melanoma of the skin, histological information could be vital to determining the prognosis of the disease. For instance, the ingression of melanocytes into the papillary dermis layer of the skin and, in particular, the depth of ingression into the papillary dermis has been correlated to the prognosis of the disease (Neville, C.D. “Melanoma: Issues of Importance to the clinician”, British Journal of Hospital Medicine, 1995). For this reason, a device that could provide histological information about an area of skin rapidly and by a non-invasive technique would be a distinct advantage.

The principal chromophores located in the skin include melanin, haemoglobin, oxy-haemoglobin and collagen. In normal healthy skin, melanin is located exclusively in the epidermis, and haemoglobin and oxy-haemoglobin are located primarily in the papillary dermis and to a lesser extent the reticular dermis. Collagen is located throughout the dermis, with the highest concentration residing in the reticular dermis. Abnormalities in the distribution of such chromophores can provide valuable information about the histology of a skin ailment and can be obtained by detecting and interpreting the distribution of different chromophores within the skin. Our application WO 98/22023 discloses a non-invasive method by which the skin colour co-ordinates and the papillary dermis thickness are determined by the analysis of light remitted from an area of skin following illumination.

Our co-pending United Kingdom patent application numbers 99 12 908 and 99 25 414 relate to advances and improvements in the determination of the concentration and distribution of chromophores within the skin. In particular, United Kingdom patent application number 99 12 908 relates to methods and apparatus by which the histology of the skin may be determined and the identification of the presence depth and concentration of chromophores within the skin. United Kingdom patent application number 99 25 414 relates to a method and apparatus for providing the information of the skin structure, more particularly, to mapping the surface of dermal papillae.

Furthermore, our co-pending United Kingdom patent application Number 00 10 888.6 relates to an apparatus and methodology for determining the distribution of chromophores within the histological layers of the skin.

Conventional methods for the diagnosis of skin ailments involve the examination of the surface characteristics of a skin lesion. In addition, and dependant on the skin condition, a proportion or entire area of a skin lesion may be surgical excised and used for histological examination under a microscope.

There are a variety of skin conditions were the provision of histological information rapidly would be a valuable adjunct to enable the efficient diagnosis of a skin ailment. In the example of a malignant melanoma, histological information could be vital to determining the prognosis of the disease. For instance, the ingression of melanocytes into the papillary dermis and in particular the depth of ingression has been correlated to the prognosis of the disease (Neville, C.D. “Melanoma: Issues of Importance to the clinician”, British Journal of Hospital Medicine, 1995). For this reason, a device which could provide histological information about an area of skin rapidly and by a non-invasive technique would be a distinct advantage.

The principal chromophores located in the skin include melanin, haemoglobin, oxy-haemoglobin and collagen. In normal healthy skin, melanin is located exclusively in the epidermis, and haemoglobin and oxy-haemoglobin haemoglobin are located primarily in the papillary dermis and to a lesser extent the reticular dermis. Collagen is located throughout the dermis, with the highest concentration residing in the reticular dermis. Abnormalities in the distribution of such chromophores can provide valuable information about the histology of a skin ailment and can be obtained by detecting and interpreting the distribution of different chromophores within the skin.

Our co-pending United Kingdom patent application numbers 99 12 908 and 99 25 414 relate to advances and improvements in the determination of the concentration and distribution of chromophores within the skin. In particular, United Kingdom patent application number 99 12 908 relates to methods and apparatus by which the histology of the skin may be determined and the identification of the presence depth and concentration of chromophores within the skin. United Kingdom patent application number 99 25 414 relates to a method and apparatus for providing the information of the skin structure, more particularly, to mapping the surface of dermal papillae.

Furthermore, our co-pending United Kingdom Patent Application Number 00 10 888.6 relates to an apparatus and methodology for determining the distribution of chromophores within the histological layers of the skin. An example of the skin illumination and remitted light detection apparatus to which the present invention relates is illustrated in FIG. 13. The skin illumination and remitted light detection apparatus has a housing 2001 onto which is mounted a display screen 2002 with a touch screen operation. A handset 2003 is stored on the housing 2001 when not in use. The handset 2003 is connected to the internal system of the equipment by a flexible metal tubing 2004 which contains a bundle of optical fibres, which transmit light from a source contained with the housing 2001, and carries signals from a detector to a computer located within the housing 2001. The apparatus is supported by castors 2005, which enable the equipment of the invention to be conveniently moved into a required location.

In use an operator 2006 removes the handset 2003 from its stored position in the housing 2001 and holds the free end 2007 of the handset 2003 against the target area 2008 of the skin of a patient 2009, as shown in FIG. 14. The operator 2006 may then select options from the touch-screen 2002 to initiate the illumination and imaging of the skin area.

The images obtained are displayed in a variety of formats on the display screen and the operator 2006 can select specific representations and view the presence of specific chromophore constituents of the skin by selecting options from the display screen 2002. The images are interpreted by a suitably trained operator and differences in the distribution of chromophores between the image obtained and the predetermined models of normal healthy skin can be visualised.

Following the imaging of the skin, the light tube 2003 is replaced within the housing and a printout of the images obtained for recording purposes.

It has been found that there are four distinct problems associated with the handset 2003 and more particularly with the nose cone, which are detailed below.

Problem 1

It is clearly visible in FIG. 14 that the nose cone is contacted directly onto the skin surface. It is during this procedure that the nose cone may be come contaminated with material associated with skin surface. The current approach to remove such material is to wipe the glass surface with an alcoholic wipe. However, such a procedure may not result in complete removal contaminants from the skin surface or the glass aperture.

Problem 2

In addition, when the nose cone is located adjacent to areas of skin, such as a finger or nose, where the skin surface is not a smooth flat surface, ambient light or stray light from the surroundings may access the detector and give rise to errors in the measurement of remitted light.

Problem 3

A further disadvantage of the current nose cone is the absence of a mechanism for controlling the pressure by which the nose cone is applied to the skin surface. This is particularly significant in, for example, situations where the apparatus is used for mapping the topology of the dermal papillae and applying too much pressure to the skin results in a flattening of these papillae.

Problem 4

A final disadvantage of the current nose cone is the requirement to provide a clinician with a clinical view. By clinical view we mean a view of the macroscopic skin surface which is a valuable adjunct to the images of light remitted from an area of skin for which the apparatus to which the present invention pertains is designed.

The present invention is concerned with overcoming the above mentioned problems or at least significantly reducing them.

The present invention relates to a simplified apparatus to and methodology for determining the concentration and/or the distribution of chromophores within an epithelial surface.

The present invention also relates to an improvement of the apparatus disclosed in our corresponding UK Application Number 0010888.6.

### BRIEF SUMMARY OF THE INVENTION

According to a first aspect of the present invention there is provided a hand-held device for the determination of the concentration and distribution of chromophores within an epithelial surface, comprising illumination means configured to illuminate an area of said epithelial surface; detection means to convert remitted light into an electrical signal; processing means configured to analyse the difference in concentration of one or more of said chromophores; and display means for displaying an output from said processing means.

By chromophore we mean any constituent of the epithelial surface having chemical groups capable of the absorption or scattering of specific wavelengths or wavelength ranges of light.

By epithelial surface we mean any epithelial tissue including, in particular, the skin, nails, the lining of the nose, rectum, vagina, mouth, ear and eye (including the corneal surfaces and retinal tissues).

It is an important feature of the first aspect of the present invention that the device is a portable hand-held device of convenient size and shape to access a number of epithelial tissue areas of varying geometry, e.g. the bridge of the nose, the surfaces of the ear etc. Furthermore a cost-effective device would be a distinct advantage.

According to a second aspect of the invention there is provided a method of interpreting the distribution of chromophores within a lesion of an epithelial tissue, said method comprising the steps of: illuminating the epithelial surface detecting the intensity of light remitted from the skin surface to form a first data set determining the variation in intensity of said remitted light across said lesion, and providing an output corresponding to the significance of the variation between the intensity of light remitted across the lesion.

The image can be obtained at one or more points over a defined distance or over a defined time.

According to a third aspect of the present invention there is provided a method of interpreting the distribution of chromophores within a lesion of an epithelial tissue, said method comprising the steps of: illuminating an epithelial surface; determining the intensity of light remitted from said lesion to form a first data set; illuminating an epithelial surface; obtaining an image of light remitted from said healthy epithelial tissue to form a second data set; and equating variations in remitted light within said first and second data sets, and providing an output corresponding to the extent of variation within or between each data set.

Also according to the present invention there is provided a skin illumination and remitted light detection apparatus, comprising a light tube defining a transparent glass aperture contactable with the skin, illumination means configured to transmit light to said light tube, detection means to detect light remitted from the skin, wavelength selection means to select the wavelength of light incident on said detection means, and illumination intensity selection means to select the intensity of light incident on the detection means, characterised in that said apparatus further comprises barrier means configured to prevent direct contact between said glass aperture and said skin.

Also according to another aspect of the present invention there is provided a skin illumination and remitted light detection apparatus, comprising a light tube defining a transparent glass aperture contactable with the skin, illumination means configured to transmit light to said light tube, detection means to detect light remitted from the skin, wavelength selection means to select the wavelength of light incident on said detection means, and illumination intensity selection means to select the intensity of light incident on the detection means, characterised in that said apparatus further comprises an ambient light exclusion means to prevent ambient light accessing said glass aperture.

Also according to a further aspect of the present invention there is provided a skin illumination and remitted light detection apparatus, comprising a light tube defining a transparent glass aperture contactable with the skin, illumination means configured to transmit light to said light tube, detection means to detect light remitted from the skin, wavelength selection means to select the wavelength of light incident on said detection means, and illumination intensity selection means to select the intensity of light incident on the detection means, characterised in that said apparatus further comprises a pressure detection means configured detect a threshold level of pressure between said light tube and the skin.

Also according to yet another aspect of the present invention there is provided a skin illumination and remitted light detection apparatus, comprising a light tube defining a transparent glass aperture contactable with the skin, illumination means configured to transmit light to said light tube, detection means to detect light remitted from the skin, wavelength selection means to select the wavelength of light incident on said detection means, and illumination intensity selection means to select the intensity of light incident on the detection means, characterised in that said apparatus further comprises a means for locating said glass aperture a defined distance from said skin surface such that a clinical view of the skin surface is obtained.

### BRIEF DESCRIPTION OF THE DRAWINGS

How the invention may be carried out will now be described by way of example only and with reference to the accompanying drawings in which:

FIG. 1 illustrates generally how apparatus according to the present invention is used;

FIG. 2 is a diagrammatic sectional view of one embodiment of the invention using a single point detector and one or more point light sources;

FIG. 3 is a fragmentary sectional view, but similar to FIG. 2, illustrating an alternative single point detector arrangement;

FIG. 4 is a block diagram illustrating the optical/electronic system incorporated in the embodiment of FIG. 2;

FIG. 5 is a view similar to FIG. 2 but illustrating a second embodiment of the invention utilising a multiple detection line array arrangement;

FIG. 6 is an enlarged perspective fragmentary view illustrating the line array shown in FIG. 5;

FIG. 7 is similar to FIG. 3 but illustrating a third embodiment of the present invention utilising a scanning mirror;

FIG. 8 is a flow chart illustrating the process by which data sets are collected and analysed;

FIG. 9 is a further flow chart providing more detailed information regarding sections 801, 802, 803/808 and 8041809 of FIG. 8;

FIG. 10 is a schematic representation of a malignant melanoma;

FIG. 11 is a graphical representation of the variations in the concentration of blood across along line 1003 of FIG. 10;

FIG. 12 is a graphical representation of the variations in the concentration of dermal melanin across along line 1003 of FIG. 10.

FIG. 13 is a perspective view of an example of the equipment to which the present invention relates;

FIG. 14 is a schematic representation of the apparatus of FIG. 15 in use;

FIG. 15 is a perspective representation of the nose cone of the apparatus shown in FIGS. 13 and 14;

FIGS. 16a and 16b are perspective representations of an example of disposable nose cones;

FIG. 17 is a schematic representation of a nose cone fitted with a disposable film cover;

FIG. 18 is a schematic representation of a transparent film coating that may be applied to the skin;

FIG. 19 is a cross-sectional view of the film shown in FIG. 20, taken along the line X-X′;

FIG. 20 is a schematic representation of a nose cone in contact with a finger;

FIG. 21 is a flow chart illustrating an example of an operational sequence to detect the presence of stray light;

FIG. 22 is a schematic representation of a film covet attachment;

FIG. 23 is a schematic representation of an alternative film attachment shown in FIG. 22;

FIG. 24 is a schematic representation of a nose cone in contact with a skin surface;

FIG. 25 is a schematic representation of a nose cone configured to detect the pressure applied to the skin;

FIG. 26 is a schematic representation of an alternative embodiment of the nose cone shown in FIG. 25;

FIG. 27 is a schematic representation of a further nose cone equipped with a mechanical means for determining the pressure with which the nose cone is applied to the skin;

FIG. 28 is a flow chart showing an example of an operational sequence configured to prevent over pressurising the skin below;

FIG. 29 is a perspective view of a nose cone with spacer legs in an upright position; and

FIG. 30 is a perspective view of a nose cone shown in FIG. 29 with the legs in a down position; and

FIG. 31 is a diagrammatic representation of a further embodiment of a portable hand-held device similar to that of FIG. 1.

### BEST MODE FOR CARRYING OUT THE INVENTION

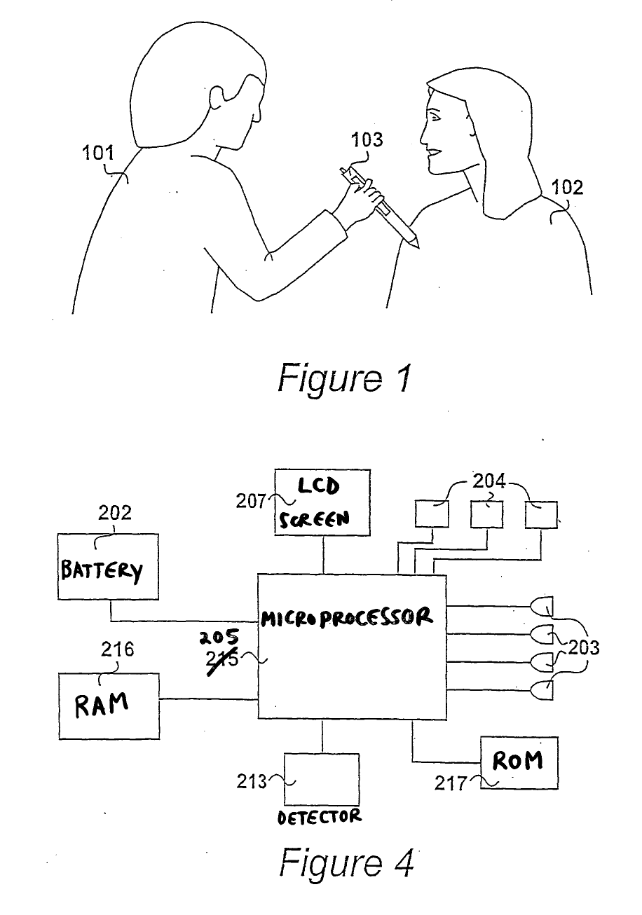

FIGS. 1 to 12

A schematic representation of an example of a device according to the present invention is shown in FIG. 1. Illustrated in FIG. 1 is a clinician 101 carrying out an examination of a patient 102 using a device 103 according to the present invention.

The device is switched on and positioned by the clinician 101 against the skin of the patient 102 at a locality or localities where the patient's skin has a visibly discernible lesion, such as a mole.

The device 103 is used to provide the clinician with a preliminary indication of whether or not the skin irregularity is likely to be malignant and require treatment or excision. The device 103 is in a form which is akin to that of a pen in that it is small and convenient to hold in the hand of the clinician 101 and is easily portable.

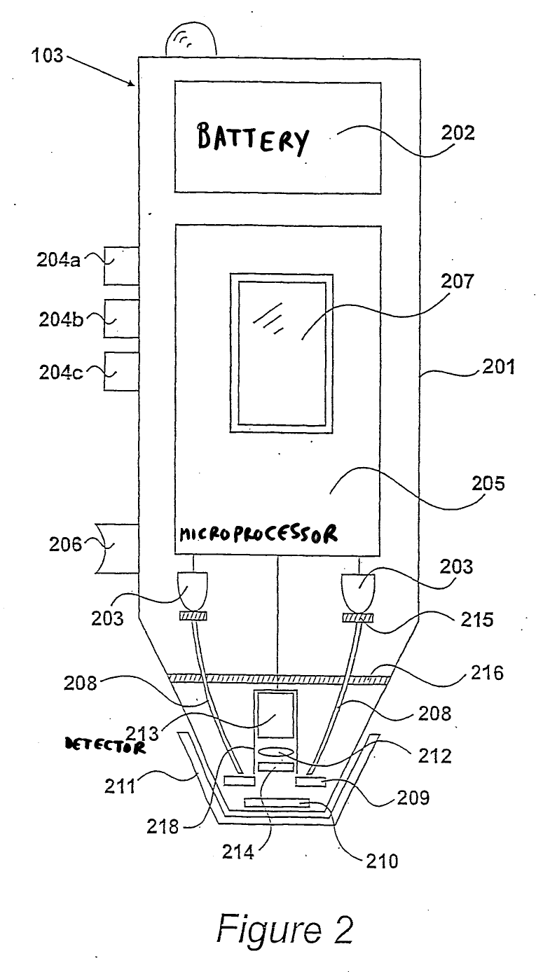

The construction of the device 103 shown in FIG. 1 will now be described in more detail and with reference to various embodiments of that device which are shown in FIGS. 2 to 7.

A schematic representation of a device according to the invention is shown in FIG. 2. The device 103 comprises a casing 201 which is adapted to be held in an operators hand and exclude ambient light from the surroundings. Incorporated within the casing is an optical system configured to provide a measure the light remitted from a small area of the skin following illumination. Furthermore, the device is provided with a microprocessor for analysing the remitted light data, interpreting the distribution and concentration of chromophores and presenting the variation in chromophore concentration on a display means.

The casing 201 contains a battery 202 which serves as the power supply for the microprocessor 205 and a series of light emitting diodes (LED's) 203. The battery is activated by depressing the on/off switch 206.

The LED's 203 are arranged in a circular, ring-like conformation. Furthermore, a series of LED's are present and each series is configured to illuminate the skin with a defined spectral profile of illumination. The selection of the series of LED's illuminated is mediated by multiplexing operation under the control of the microprocessor 205.

Light emitted by the LED's passes through a band pass filter 215 which selects the wavelength/wavelength range of light, which is subsequently directed towards the skin along the optical fibre bundle 208.

Light emitted from the terminal of the optical fibre bundle 208 passes through a first polarisation filter 209 and illuminates the skin through the glass aperture 210.

Light remitted by the skin surface passes through a second polarisation filter mounted such that the angle of polarisation is at ninety degrees to that of the first polarisation filter. The polarisation filters prevent light reflected from within the device or the skin surface from accessing the detector and obscuring the detection of light remitted by the skin surface. The entire detection system is mounted within a cover 218 to prevent ambient light in the nose cone accessing the detector. Furthermore, the entire nose cone section is isolated from the illumination assembly by an opaque screen 216 to prevent stray light from the LED illumination array accessing the detector 213.

Light traversing the second polarisation filter 214 is focused by a lens 212 and detected by a point detector 213. The detector comprises a silicon photodiode or phototransistor which is configured to convert the intensity of light detected into an electrical signal which is fed into the microprocessor 205 for further analysis.

The data collected by the detector 213 is processed by the microprocessor 205, analysed and an appropriate display is presented on the LCD screen 207. In addition, to alert an operator, a coloured light 215 is lighted in response to a significant result obtained.

The operative end of the casing 201 carries a thin transparent cover 211 which in use is pressed against the skin of the patient. The part of the nose-cone in line with the window 210 is transparent to the light being emitted by the respective LED's 203 and light entering the device from the skin surface.

The nose cone may optionally comprise a series of homogenous grey-scale calibration patches to calibrate the image of remitted light obtained. A detailed description of an example of such a calibration procedure can be found in our co-pending UK Patent Application Number 00 10 888.6. The construction, and alternative constructions for the nose-cone are described in more detail in our co-pending UK Patent application.

In use, the operator identifies the lesion of interest, places the glass aperture directly against the skin surface and initiates the collection of data by depressing the activation switch 206. Depressing the switch 206 activates the microprocessor 205 and initiates a program sequence of obtaining a data sets corresponding to light remitted by the skin at a single point.

The microprocessor program initiates the illumination different LED series, one series at a time, and detecting the light remitted at each illumination step. The microprocessor measures and stores remitted light signal from the detector in the memory.

The device is slowly moved across the lesion and numerous data sets corresponding to the spectral characteristics of remitted light at each point traversed by the device is detected and stored in the memory. The speed of movement of the device should be sufficient to enable data sets to be obtained at each point, although the speed of detection of the data is not thought to be a limiting factor, rendering the speed of traverse less critical.

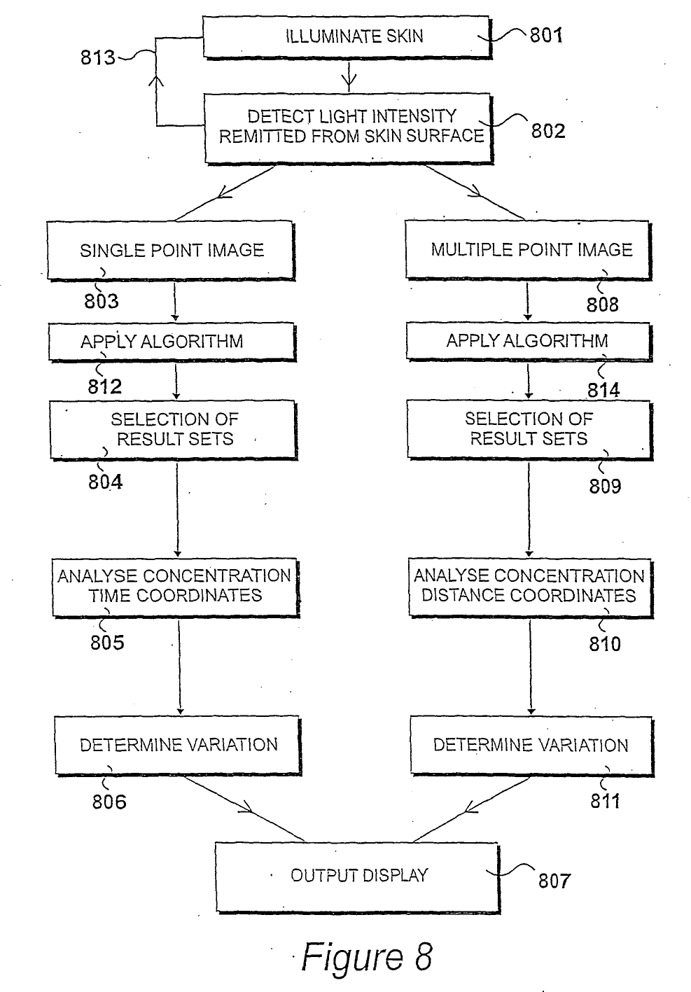

The process by which the data sets are obtained and converted into a result set are discussed in more detail in reference to FIG. 8. The operator can select different results sets for display on the display screen 207 by selectively depressing one of the switches 204a, 204b and 204c.

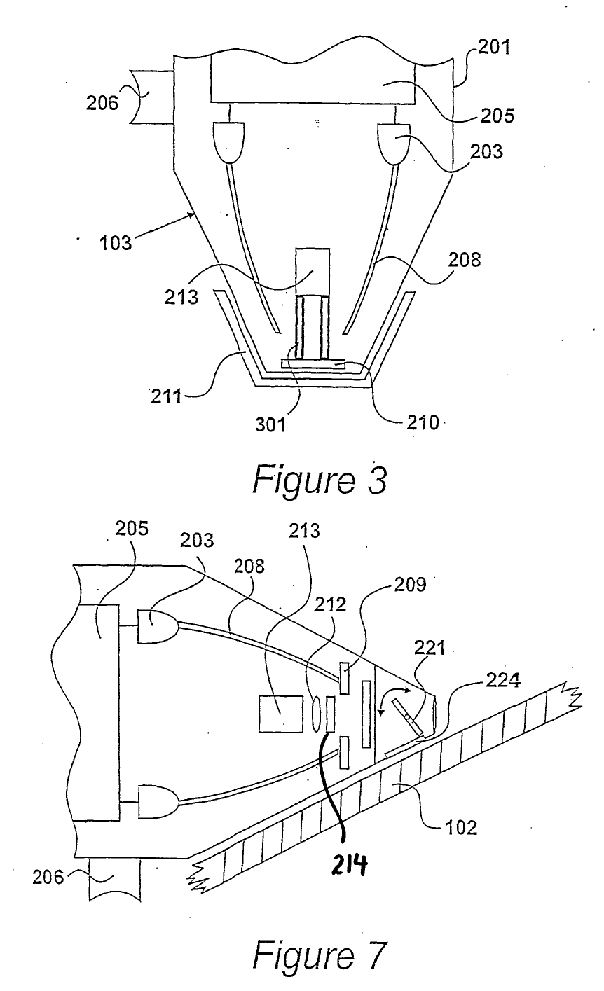

FIG. 3 illustrates an alternative construction of the operative end of a device according to the invention. Light emitted by the LED's 203 is transmitted through the glass aperture 210 via the optical fibre bundles 208 in the conventional manner. No polarisation filters are required in this embodiment as fight emitted by the termini of the optical fibre bundles 208 cannot access the detector 213 directly due to the presence of an opaque remitted light channel 301. As no direct access of illuminating light is enabled, light entering the opaque remitted light channel 301 emanates solely from remittance from the skin surface contacted with the nose cone 210.

As with the device shown in FIG. 3, the data obtained by the detector 213 is transmitted to and processed by the microprocessor 205.

A schematic representation of the essential components of the device are shown in FIG. 4. Corresponding reference numerals are used to identify like or corresponding parts.

The system is powered by a battery 202 which in turn is controlled by an on/off switch 206. The microprocessor 205 comprises random access memory 216 and read only memory 217 segments as in standard microprocessor units. The microprocessor 205 is activated by the depression of switch 206, which initiates the illumination of the skin by the LED array 203. The detector 213 images the intensity of light remitted from the skin surface and, generates a signal that is conveyed to the microprocessor 205.

The data obtained from the detector is stored in the microprocessor memory, subsequently analysed and displayed on the display screen 207.

The devices illustrated in FIGS. 2 and 3 detect light remitted from a single spot on the patient's skin, the device then having to be moved across the patient's skin in order to build up useful data of the target area.

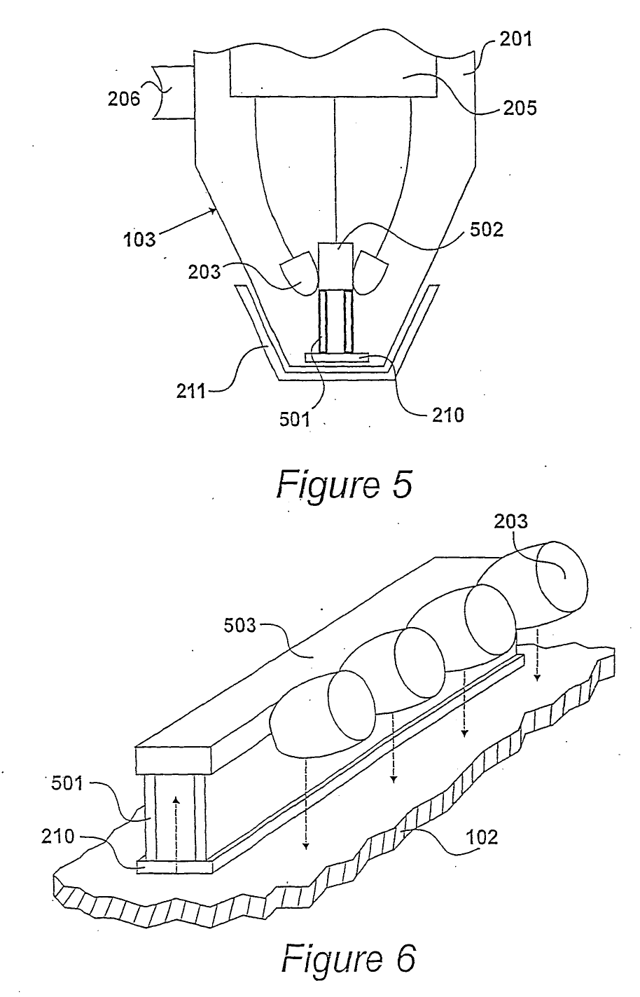

The embodiment shown in FIGS. 5 and 6 is basically the same as the embodiment shown in FIGS. 2 and 3 but with the difference that in the embodiment of FIGS. 5 and 6 a line detection array 502 is used to record the light remitted from the patient's skin rather than a single spot detector. The device is shown in cross section in FIG. 5 with the line detector 502 extending into the paper. The line array may be of any length although the range one to fifty millimetres is preferred with twenty millimetres most preferred.

The detector comprises a series of single point detectors arranged in a line. A linear diffuse LED array 203 is mounted onto the linear detector array. As with previous devices, one or more LED series (each series corresponding to a specific wavelength or wavelength range of illumination) are present, each series configured to provide illumination of a specific wavelength.

The device shown in FIG. 5 comprises a series of remitted light channels 501 illustrated in perspective in FIG. 6. The light channels are arranged such that there is one light channel per single point detector of the detection array. The detector comprises a silicon base 503 on which LED's 203 are mounted (see FIG. 6).

Light emitted by the LED's 203 accesses the skin 102 through the window 210 to produce a line of illumination. Remitted light from the skin 102 accesses the detector 502 through the channels 501. Consequently, each detector detects the intensity of light remitted at a point along the line of illumination.

In this embodiment, and in common with the embodiment shown in FIG. 3, there are no polarisation filters as the reflection of light into the detector is excluded by the opaque remitted light channels 501.

With this arrangement the device is placed across a lesion and the intensity of remitted light is dependant on variations in chromophore concentration with distance along the length of the detection array 502. Alternatively, the detection array 502 is scanned across the patient's skin in the vicinity of the target area to build up an image in which variations in the intensity with time (corresponding to the distance scanned across the lesion) are recorded.

As indicated earlier the device is shown in FIGS. 2 and 3 it is necessary for the operator/clinician to physically move the operative end of the device across the patient's skin.

FIG. 7 illustrates a modification to the device which would obviate the necessity to do this by providing a mechanical means by which the point of illumination is scanned across the lesion.

The device illustrated in FIG. 7 is essential equivalent to that of FIG. 2. The device has a casing 201, a microprocessor 205, LED's 203 that transmit light along optical fibre bundles 208, a detector 213 and first and second polarisation filters 209 and 214. The mechanical scanning of a skin surface is controlled by a rotatable mirror 221. The mirror pivots about a points such that the illumination is scanned across the skin surface 102. Remitted light from the surface is correspondingly reflected towards the detector 213.

This mirror would be mounted and driven in such a way that its angle to the axes of the device 201 would vary typically between, for example, fifty degrees and forty degrees, the medium position being forty-five degrees.

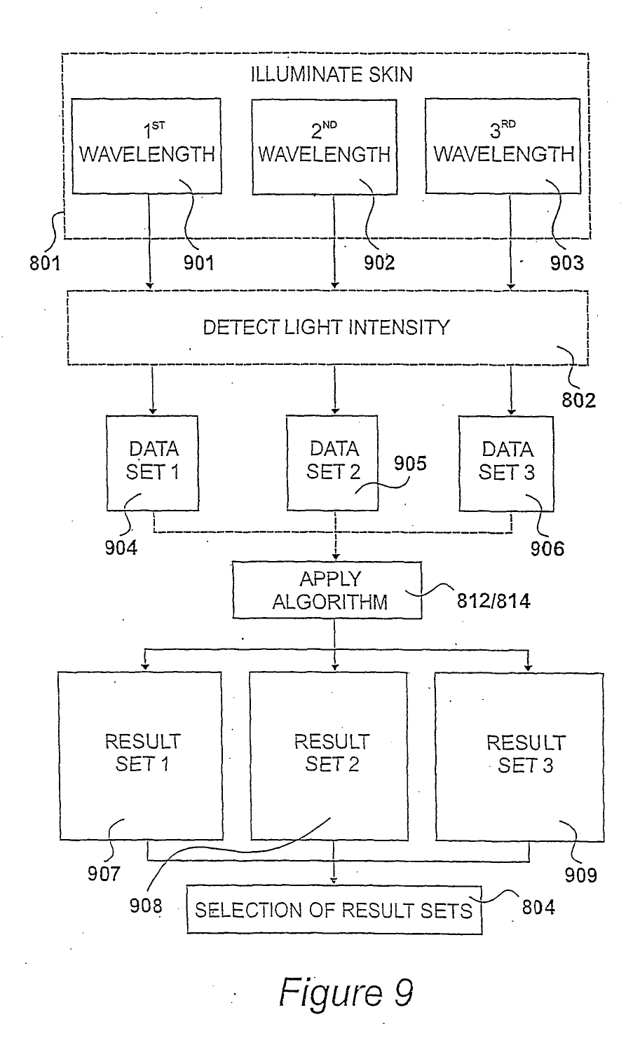

In use the skin of the subject 102 is placed directly adjacent to the glass aperture 224 of the device. Preferably, the glass aperture 224 is pressed against the skin 102 such that ambient light from the surroundings cannot access the detector. The lesion is then scanned and the data collected and analysed as discussed in reference to FIGS. 8 and 9.

Various further modifications could be made to the devices previously described with reference to FIGS. 1 to 7.

For example instead of simply selecting a single LED to illuminate the patient's skin all the LED's series carried in the device could be energised in turn by a multiplexing arrangement. This would provide a sequential illumination of, for example, red, blue and infrared wavelengths. The precise wavelengths selected will depend on the chromophores within the skin under investigation.

In addition, the skin could be illuminated by any suitable means, for example, a light bulb provided with a bandpass filter, to select the wavelength, or diffracted through a prism to enable the selection of wavelength constituents, incandescent lamps with band pass filters, light emitting polymers or other low power fluorescent devices.

In the following description of FIGS. 8 to 12, the term “data set” is used to specify the intensity-time/distance data of the remitted light. The term “result set” is used to specify the analysed data sets which provide information regarding the distribution and concentration of chromophores derived from the analysis of the the above defined data sets.

The process by which the variation in the concentration of a chromophore within a skin lesion is determined is illustrated in FIG. 8. The initial step 801 involves the illumination of the skin with light of the desired wavelength and intensity. The wavelength of light selected will be a specific wavelength or wavelength range. For example, near red or infra-red wavelengths can be used to determine the distribution of collagen within the skin surface and red light wavelengths are required to determine the distribution of haemoglobin (blood) within the skin surface.

The remitted light is detected 802, converted into an electrical signal and the skin re-illuminated at a second wavelength/wavelength range 813 and the intensity of light remitted following the illumination at a second wavelength is detected. The intensity of remitted light recorded is stored by the microprocessor 206 as separate data sets.

The subsequent processing of the data sets obtained will depend on whether a single point detector is used, as described in reference to FIGS. 2, 3 and 7, or a multiple point image detector is used, as described in reference to FIGS. 5 and 6 and our co-pending United Kingdom application number 00 10 888.6.

In the case of single point detectors 803, the data set comprises a series of intensity readings obtained over time. The point of illumination and the point of remitted light detection is traversed across the image, preferably by manually moving the device across the lesion or by mechanical means as described in reference to FIG. 7. Consequently, variations in the concentration of chromophores across the skin lesion results in corresponding changes in the intensity of light remitted from the skin surface. Each data set will correspond to intensity time data obtained at a specific wavelengths/wavelength ranges of illumination.

An algorithm 812 is applied to the data sets which determines the concentration and distribution of specific chromophore constituents within the skin. The algorithms applied are described in detail in our co-pending patent applications WO 98/22023 and United Kingdom patent application numbers 99 12 908 and 99 25 414. The intensity of the light remitted from the skin over time stored in each data set is used to create a series of results sets using the above mentioned algorithms. Each result set corresponds to the variation in the concentration of each chromophore recorded over time.

The appropriate results sets are selected 804 which correspond to the particular chromophore distributions under investigation.

Following the selection of the results sets, the variation in the concentration and distribution of the chromophore over time within each result set is individually analysed 805. The extent and significance of the variation is determined 806 and a significance output is displayed on the output display 807.

The output display 807 is designed to signal to an operator whether the variations in concentration of one or more chromophores is sufficiently indicative of an abnormality. The output enables an operator or clinician to contemplate further action necessary, such as the excision of the lesion.

The output display 807 may take a variety of forms. In the simplest embodiment, the output display is a single LED which lights if an abnormality is detected. Alternatively, a series of LED's, whereby the number of which light corresponds to the degree of variation detected could be used. A further alternative is the display of the output as a number presented on the display 207. The value of the number corresponding to the degree of variation within the results set.

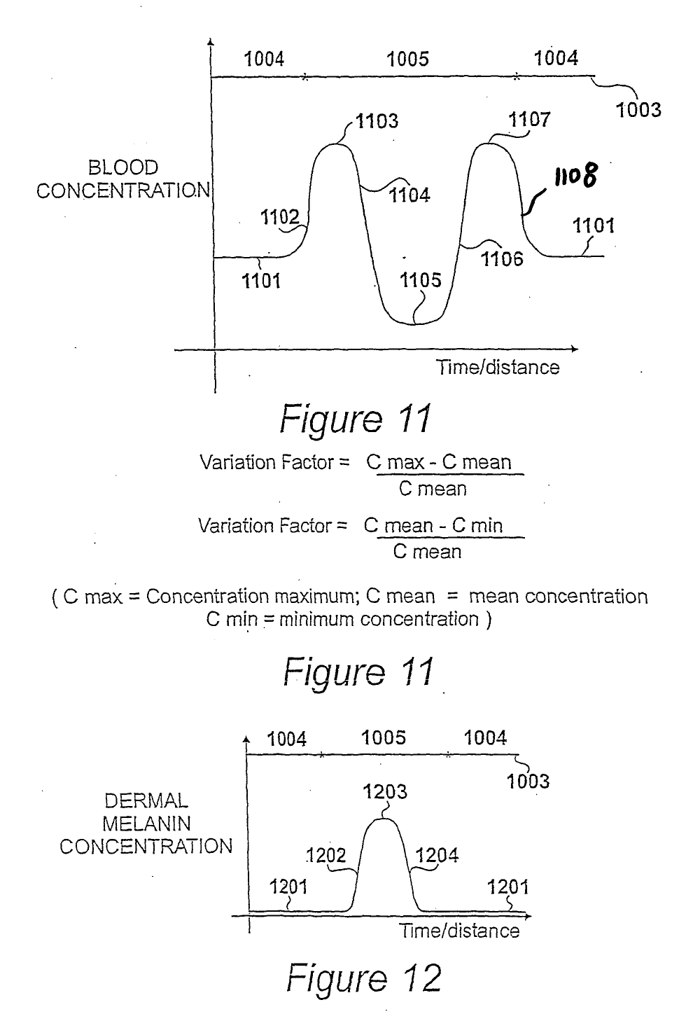

In situations were the user wishes to view the mapping of the chromophores over the lesion a graphical representation, such as that illustrated in FIGS. 11 and 12, could be viewed on the screen 107. The operator will be able to select different chromophores by, for example, depressing buttons such as 204a, 204b and 204c of the device shown in FIG. 2. Alternatively all the chromophores may be displayed simultaneously.

A final display option is provision of display relating to the likely structure of the skin as interpreted by the microprocessor following programming to correlate the distribution and concentration of chromophores to specific skin structures.

In the case of a multiple point imaging device 808, where an image is obtained across a lesion, the intensity variations across a section taken through the lesion is used to provide information relating to the concentration and distribution of chromophores as a function of distance across the lesion.

Individual data sets corresponding to images obtained at specific wavelength/wavelength ranges are stored.

As with the single data point intensity-time data sets, an algorithm is applied the intensity-distance data plots to convert the data obtained into the distribution and concentration of specific chromophores within the skin. The resultant chromophore concentration and distribution information is stored as a result set.

The appropriate result sets are selected 809, analysed 810 and the variation in chromophore concentration across the lesion determined 811. As for the single data point apparatus the variation in chromophore concentration in one or more result sets is displayed on the output display 807.

The selection of remitted light results sets is illustrated in more detail in FIG. 9. The skin is illuminated at three wavelengths 901, 902 and 903 respectively. The intensity of the light remitted across the lesion from wavelengths 901, 902 and 903 provides data sets 904, 905 and 906 respectively.

As previously described, an algorithm is applied to the data sets to provide a series of results sets, for example 907, 908 and 909. Each result set relates to the variation in concentration and distribution of a respective chromophore across the lesion. For example, result sets 907 to 909 could correspond to dermal melanin, blood distribution and collagen distribution respectively.

These variations in the concentration of chromophores within an epithelial tissue, such as the skin, is indicative of specific abnormalities.

Of the three result sets provided, one or more of these result sets is selected 804 and subsequently analysed for variations in the concentration of a chromophore 805. The selection of results sets is a matter of choice for the clinician or may be predetermined for specific skin conditions.

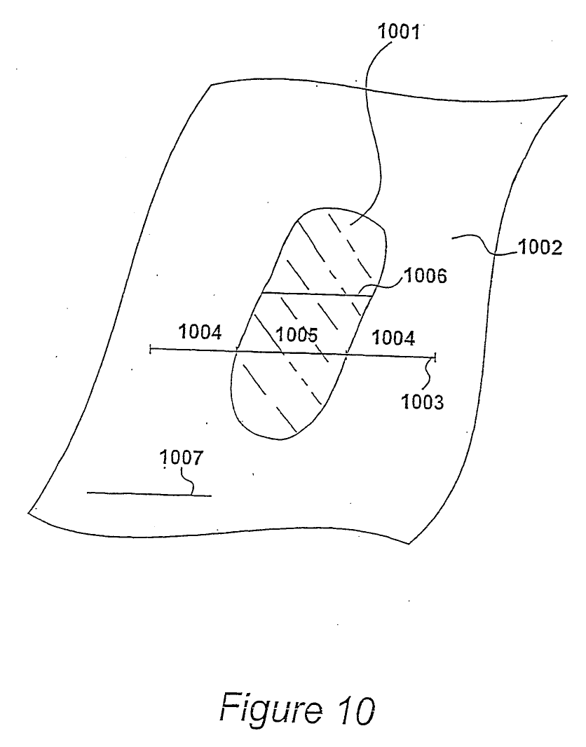

A schematic representation of a malignant melanoma lesion is shown in FIG. 10. The malignant melanoma 1001 is surrounded by normal healthy skin 1002. An image data set is obtained by determining variations in chromophore concentration along line 1003. Line 1003 has sections 1004 which traverse healthy tissue and 1005 which traverses the lesion. In the case of a single point device the point of illumination and detection is moved along line 1003. In the case of a multiple point detector, the data along line 1003 is selected from an image for further analysis.

Alternatively, line 1006 provides a lesion data set which is compared with a second data set obtained at line 1007 which corresponds to healthy skin. A comparison between the data sets corresponding to line 1006 and 1007 provides an indication of abnormality within the lesion relative to the normal skin.

Concentrating on a single result set obtained along line 1003, an example of a result set is illustrated graphically in FIG. 11. FIG. 11 details the variation in concentration of blood along line 1003.

It has been found that, in malignant melanomas and other cancers, the blood vessels are excluded from the centre of the tumour and concentrate about the periphery of the lesion. This is known as an erythematous blush. The identification of this feature is hence, indicative of a malignant melanoma.

The graph shown in FIG. 11 shows a plot of time/distance (dependant on whether a single point or multiple detection device is used) on the x co-ordinate and blood concentration on the y co-ordinate.

In normal skin sections 1004, the concentration remains relatively constant 1101, which indicative of a homogenous distribution of blood. At the periphery of the lesion 1005, the concentration rises 1102 to a concentration maximum (Cₘₐₓ) at 1103. The intensity falls at 1104 to an concentration minimum (Cₘᵢₙ) at 1105. The fall in concentration corresponds to the central regions of the melanoma were blood perfusion is minimal.

As the periphery of the lesion is approached again, the concentration rises at 1106 to a second maximum 1107. The concentration declines at 1108 to the level of normal skin 1101.

As variations in blood concentration of the skin, is indicative of abnormality, the intensity data is used to calculate a variation factor, the value of which relates to the extent of variation within the result set.

Examples of equations by which a suitable variation factor is calculated are also shown in FIG. 11. The first equation correlates the variance between the concentration maximum (Cₘₐₓ) and the mean concentration (Cₘₑₐₙ) of the result set. Hence, the value of the variation factor is dependant on the difference between the concentration maxima and the mean concentration value. For a given chromophore, a threshold value above which the variation is considered significant is set or alternatively, range values of the variation factor can be defined to provide an indication of the extent of variation within the data set.

A second equation shown in FIG. 11 works on similar principles and equates the concentration minima (Cₘᵢₙ) with the mean concentration (Cₘₑₐₙ).

A variety of alternative statistical measures could be applied to determine the level and significance of variations.

FIG. 12 shows a second result set corresponding to dermal melanin along line 1003 of FIG. 10. In malignant melanomas, the presence of melanin that has penetrated into the papillary dermis is a significant prognostic factor. In normal skin the amount of melanin within the dermis is virtually negligible and consequently, the detection of any melanin in the dermis can be an indicator of malignancy.

In FIG. 12, the dermal melanin concentration, as identified by the remitted light intensity, is virtually negligible in normal skin. However, within the lesion, the concentration of dermal melanin rises at 1202 to a maximum at 1203 and declines at 1204 back to the negligible level 1201.

In this case, a binary equation can be applied as the dermal melanin is either present or absent. Alternatively, a variation factor which equals the concentraLion maximum can be provided as an indicator of the level of penetration into the derrriis.

The discussion of FIGS. 11 and 12 provides an example of two result sets which may be provided by an apparatus of the invention. A single result set or multiple result sets corresponding to numerous chromophores may be obtained and analysed.

In particular, it has been found that, following the assessment of one hundred and thirty eight lesions in a clinical trial using images obtained by a SIAscope device (as described in our co-pending UK Patent Application Number 00 10 888.6), in lesions of diameter of greater than six millimetres, the combination of blood displacement in the form of an erythematous blush and melanin in the dermis provided a correlation with histological examination. Upon statistical analysis, the sensitivity was found to be 91.3% and the specificity of 77.3%. Consequently, the presence of dermal melanin and blood displacement within a lesion of diameter of greater than six millimetres indicates that the lesion is four times more likely to be a malignant melanoma. Conversely., a lesion of less than six millimetres diameter and with no detectable melanin in the dermis or blood displacement is nine times less likely to be a malignant melanoma

The actual data of the trial is shown below in Table 1.

TABLE 1
the identification of malignant melanoma and non-melanomas
using a test of three criteria - melanin in the dermis, blood
displacement and lesion diameter of less than 6 mm

| | Melanoma | Non-melanoma | Totals |
|---|---|---|---|
| Test positive | 21 | 26 | 47 |
| Test negative | 2 | 89 | 91 |
| Totals | 23 | 115 | 138 |

Alternative chromophores and properties of the skin that can be analysed include the mapping of the dermo-papillary junction, total melanin distribution and keratin

It will also be appreciated that the present application is not limited to the skin and the distribution of chromophores within any epithelial surface could be determined by the devices and methodology described in the present application and our co-pending United Kingdom patent application numbers 99 12 908, 99 25 414 and 00 10 888.6

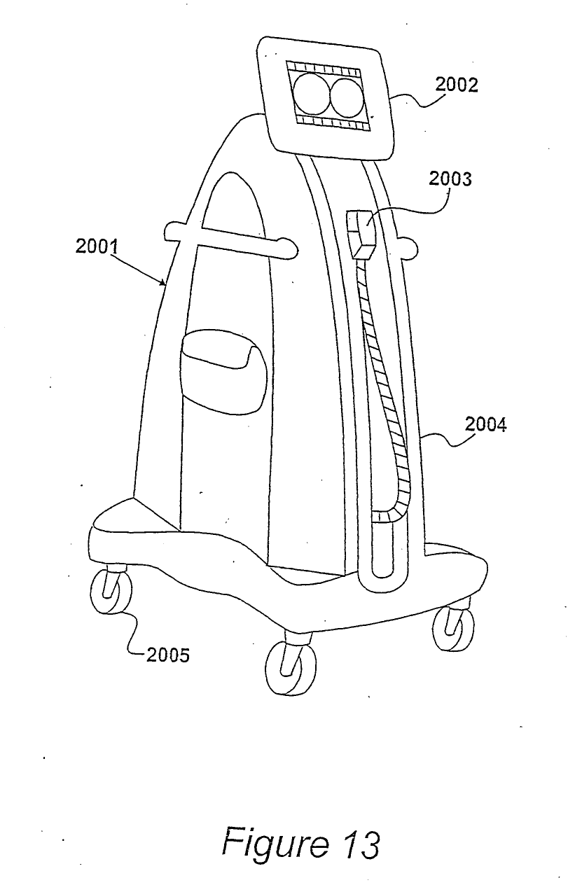

FIGS. 13 to 30

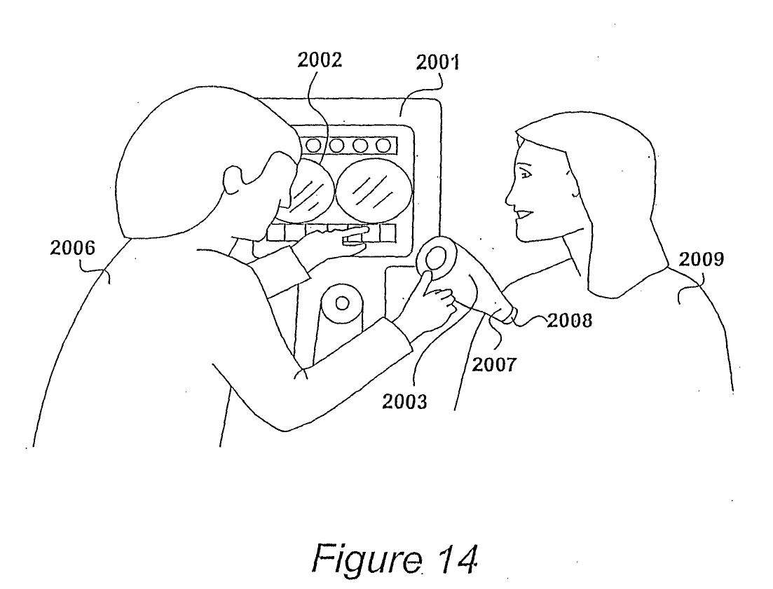

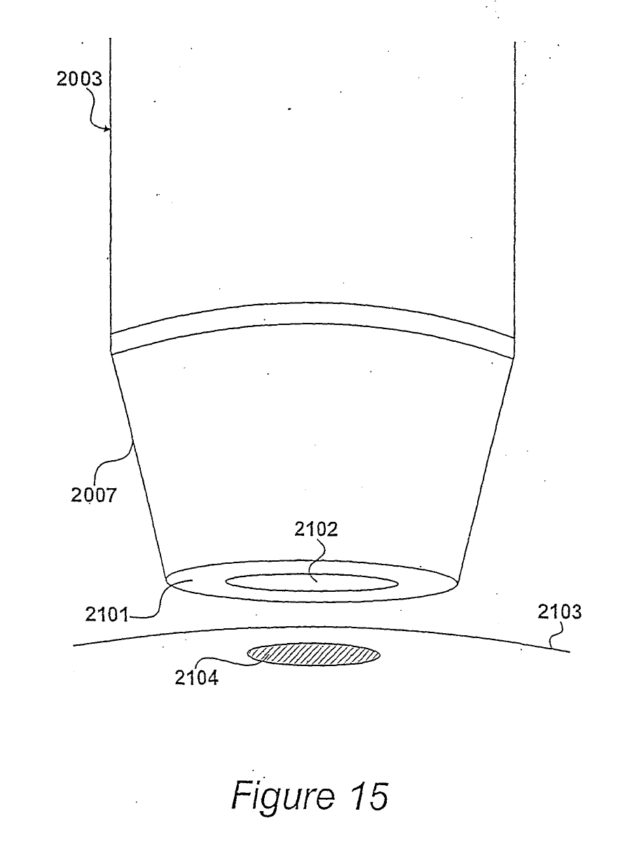

FIG. 15 shows a schematic representation of a light pipe 2003 having a nose cone 2007 with a transparent glass aperture 2102 defined by the nose cone ending 2101. Illumination from the source, is transmitted from a source located within the housing 2001 to the light pipe 2003 (see FIGS. 13 and 14) and illuminates the skin 2103 through the transparent glass aperture 2102. Also shown within FIG. 15 is a lesion 2104, for example a mole, in which the distribution of chromophores is to be examined.

To obtain an image, the skin 2103 is contacted directly against the aperture 2102. Loose skin and material on the skin surface such as, for example, skin oils, creams etc, leaves a residue on the glass aperture 2102 and which, if not removed, will affect the quality of subsequent images.

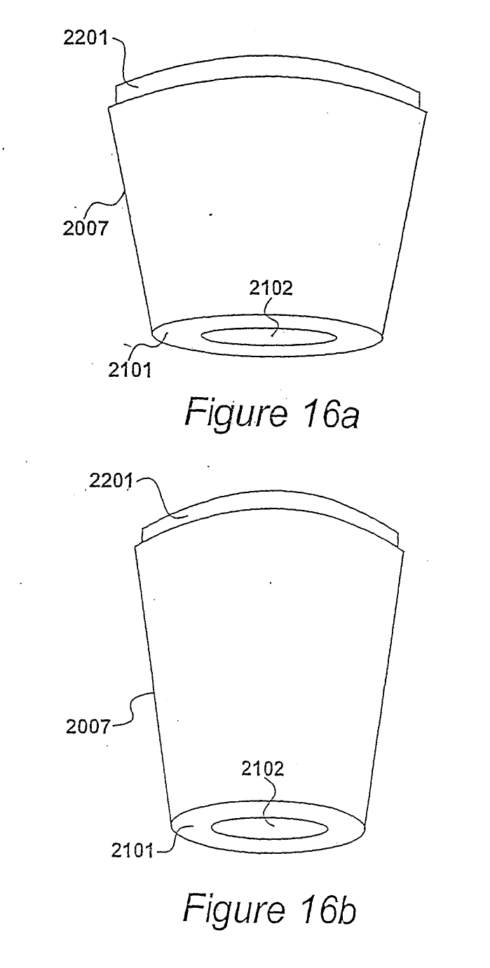

A nose cone according to the present invention is illustrated in FIGS. 16a and 16b. FIG. 16a illustrates a schematic representation of a disposable nose cone 2007 with a nose cone ending 2101 incorporating a glass aperture 2102. The nose cone 2007 illustrated in FIG. 16a comprises a male connection member 2201 which is receivable within the handset body to form a resistive fit to secure the nose cone in position. FIG. 16b illustrates another disposable nose cone similar to that shown in FIG. 16a with the exception that the nose cone body 2007 is elongated with a nose cone ending 2101 and a glass aperture 2102 of smaller dimensions to the corresponding nose cone illustrated in FIG. 16a.

To accommodate the differing dimensioned nose cone, a means is provided to adapt the detective field of the detector to accommodate the appropriate sized glass aperture. This is achieved in the present embodiment by the provision of an electrical contact on the nose cone such that, upon attaching the nose cone 2007 to the handset 2003, a contact is made with a second electrical contact provided on the handset 2003. The contact will be configured such that each different dimensioned nose cone interacts with a specific electrical contact on the hand set. Upon electrical contact between a contact on the nose cone and a contact on the handset, the lens which focuses the light remitted from the skin onto the detector is automatically moved to a predetermined position which adapts the detective field of the detector to correspond with that of the aperture of the nose cone fitted. Consequently, a variety of nose cones of differing dimensions are provided enabling the selection of different image areas.

Alternatively, the lens may be repositioned by a mechanical means, wherein the nose cone carries a probe which, upon location of the nose cone on the handset, is received by the receptacle which moves a slidably mounted lens to the required position corresponding to the length of the probe. Each different dimensioned nose cone will possess a different length probe which determines the final lens position and hence ensure correct focus of the image field of the detector within the handset onto which it is mounted.

The desired nose cone is provided within a sealed bag from which it is removed and mounted onto the handset. Upon use, the nose cone is contacted directly with the skin surface and the skin imaged as described in our previous applications. Following use, the nose cone 2007 is detached from the handset and discarded. For subsequent images, a second clean nose cone is attached to the handset.

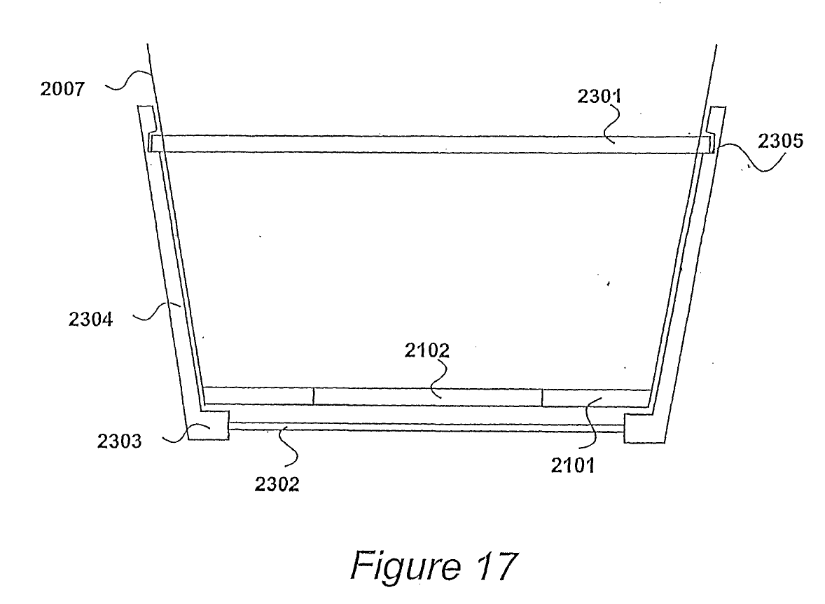

An alternative embodiment of the present invention is illustrated in FIG. 17. Attached to a nose cone 2007 is a transparent film 2302, which forms a covering over the nose cone ending 2101 and the transparent glass aperture 2102. The film serves as a physical barrier between the glass aperture 2102 and the skin and thus prevents contaminants on the skin adhering to the glass. The film 2302 is mounted taught within a plastic clip 2303 which comprises an arm 2304 which extends adjacent to the external surface of the nose cone body 2007. In the preferred embodiment, the nose cone 2007 is provided with an annular lip 2301 which extends about the circumference of the nose cone. The arm of the plastic clip 2304 comprises a recess 2305 configured to receive the annular lip 3231 of the nose cone 2007, such that the plastic clip 2303 and the transparent film 2302 mounted therein is held flush with the nose cone end 2101 and the glass aperture 2102.

The film 2302 is preferably prepared from material which is uniformly transparent to light of visible and infra-red wavelengths. Examples of suitable materials would include polyethylene, polyesters, polypropylene, polystyrene, PBDF and polyvinylchloride. The plastic film may also be coated with an adhesive to improve the adherence of the film to the skin.

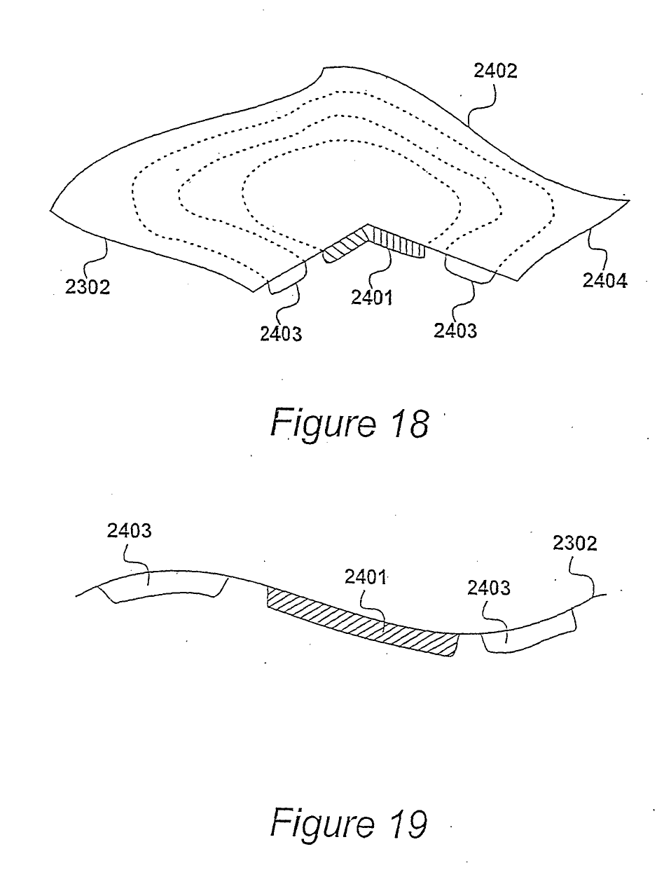

An example of an alternative film that may be incorporated into the clip illustrated in FIG. 17 is shown schematically in FIG. 18. Located on a first side of the transparent film 2302 is a second layer of an optical effective index matching oil 2401 within a defined area 2402 which corresponds to the area of the glass aperture 2102 of the nose cone 2007. In the preferred embodiment the optical matching index oil is Heine Mineral Oil although any suitable optical index matching oil would suffice such as olive oil or ultrasound coupling gel. In use the optical matching oil reduce reflections from the skin surface. The oil coats the skin on contact diffusing into cracks and abrasions on the skin surface and reducing optical inhomogeneities due to the skin topology.

Also illustrated in FIG. 18 is a further layer of adhesive 2403 of defined area 2404 which encircles the optical index matching oil area 2402. This arrangement provides for securing the film 2302 to the skin surface of a subject providing an area of optical index matching oil 2402 of comparable size to the glass aperture 2102 such that, upon illumination, light incident from the skin encounters a layer of optical index matching oil prior to contacting the surface of the skin.

FIG. 19 shows a cross-section through the film illustrated in FIG. 18 along the lines X-X′. The film 2302 has, mounted on a first side, a second layer of optical index matching oil 2401 surrounded by a further layer of adhesive 2403. As previously described, the film is contacted with the skin via the first side, upon which the second layers of optical index matching oil and adhesive are mounted, and the second side is contacted with the glass aperture preventing contaminants accessing the glass aperture of the nose cone.

Alternatively, the transparent film could be applied directly to the skin of the patient and the nose cone of the handset located against the exposed side of the film to image the area of skin. The film may be secured to the skin by a layer of adhesive. In addition, a layer of optical index matching oil could be provided as previously described with reference to

FIGS. 18 and 19.

The films incorporated in the present invention could also comprise a bar code which can be read by a bar code reader mounted within the handset or by the system intended for measuring the light remitted from the skin itself. Consequently the image can be correlated with a specific patient by the bar code for recording purposes. Such a system, could also be used to prevent re-use of a film and hence, cross contamination with material collected from the skin during a previous image process. Example of alternative datamarkings which can be used instead of bar codes include snowflake markings, alphanumeric codes or various forms of optical characters.

Furthermore, the film can be marked with a medical pen to identify areas of a lesion which may be excised. For example, in the case of a malignant melanoma, the images obtained by the apparatus of FIGS. 13 and 14 will indicate the distribution of melanin beneath the surface layers of the skin. Consequently, it may be apparent that a larger area of the lesion requires removal compared to what is evident by a surface examination. A clinician will be able to mark or transfer a mark of the area to be excised onto the film which is subsequently used as a guide to a surgeon when removing the lesion.

The following section will describe examples of embodiments of the invention designed to address the problem of stray light accessing the detector.

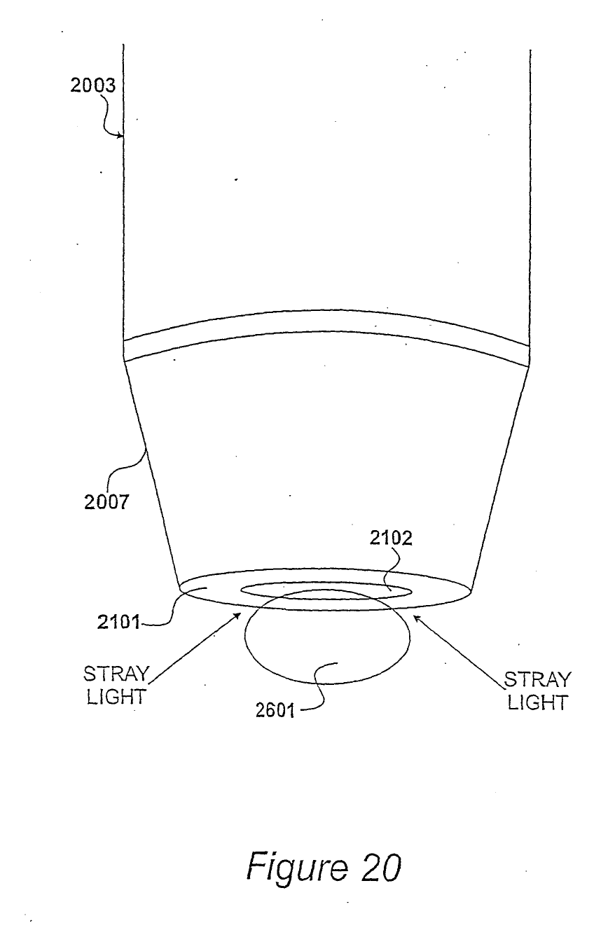

A schematic representation of an example of apparatus according to the invention in use imaging a skin surface of substantial curvature is illustrated in FIG. 20. The light pipe 2003 comprises a nose cone 2007 which further houses a nose cone end 2101 with a glass aperture 2102 mounted therein. Located adjacent to the glass aperture is a finger, represented by the object 2601. The finger 2601, by virtue of the size and curvature, does not form a complete contact with the glass aperture and consequently stray light (or ambient light from the surroundings) accesses the detector increasing the background intensity and obscuring the image of light remitted from the skin. This makes the interpretation of the image less accurate and, in situations where the light remitted from the illuminated area of skin is low, the remitted light may be undetectable relative to the intensity of stray light accessing the detector.

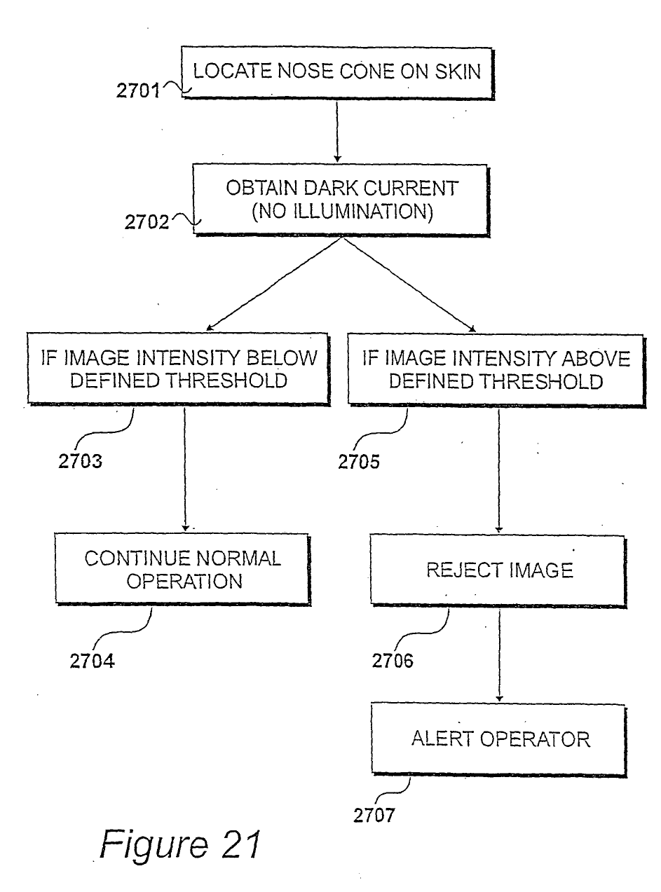

To prevent images being obtained in a situation where too much stray light is present a safety operational sequence is incorporated into the operation of the apparatus to which the invention relates. The operational sequence is illustrated in FIG. 21.

The light pipe is removed from the apparatus and located on the desired area of skin 2701. An image is recorded in the absence of incident illumination 2702. The intensity of the image detected will depend on two factors, namely the dark current of the detector (which is known during normal operation) and the presence or absence of stray light accessing the aperture. Consequently, the intensity of the image at one or more points on the detector is set to a predetermined threshold level of stray light considered acceptable. If the image intensity at one or more points is below the defined threshold 2703 the normal imaging process continues 2704. If the image intensity at one or more points is above a predetermined threshold level of illumination 2705 the image will be rejected 2706 and the operator alerted by an alarm or visual message 2707 to signify that there is insufficient contact between the desired skin area and the glass aperture.

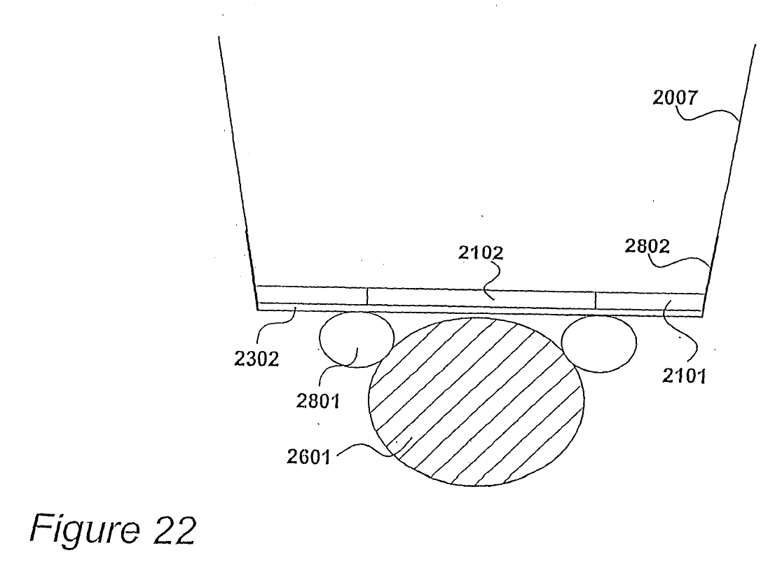

An example of an embodiment of the invention configured to prevent stray light accessing the detector is shown in FIG. 22. The nose cone 72007, with glass aperture 2101 defined by nose cone end 2102, is contacted with a finger, represented by oval object 2601. Located in between the nose cone ending and the finger 2601 is a transparent film 2302, which prevents contaminants from surface of the finger contacting the glass aperture. The transparent film 2302 is fixed to the nose cone by an adhesive coating 2802. A circular deformable foam ring 2801 is attached to the film such that stray light from the surrounding is blocked from accessing the glass aperture 2102. Any suitable deformable and optically opaque material would surface in place of the foam ring 2801, suitable examples of which include pigmented silicone rubber and visco-elastic polymers such as a material known as “silly puty” or Plasticine^{RTM}.

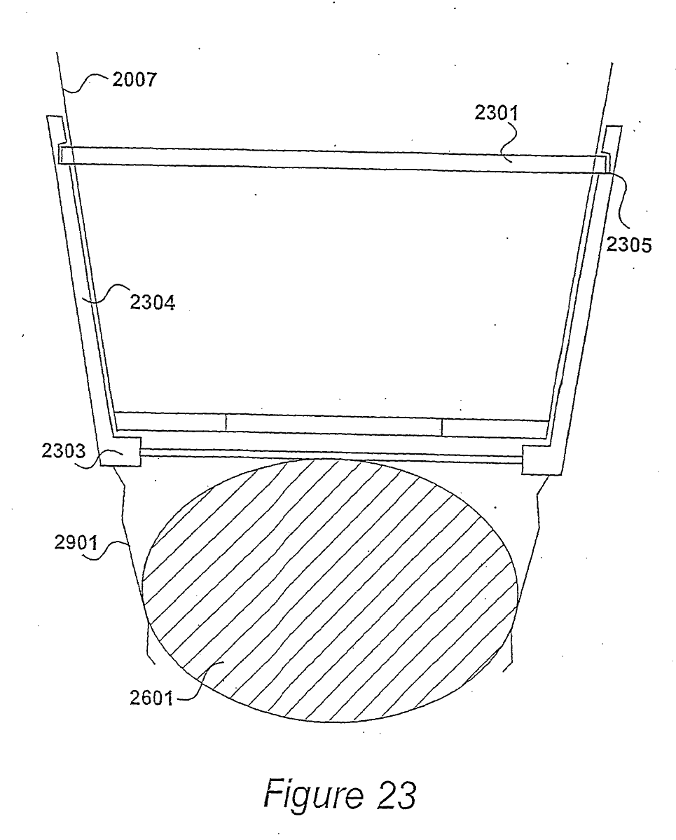

An alternative embodiment of the invention is shown in FIG. 23. A nose cone 2007 is provided with an annular lip 2301. A finger 2601 is orientated adjacent to the glass aperture 2102 with a transparent film located in between. The transparent film 2302 is mounted within a plastic clip 2303 which receives the nose cone end 2101 and is clipped into place by a groove 2301 which receives the lip 2301, as previously discussed with reference to FIG. 17.

An opaque curtain 2901 extends from the clip to associate with the finger 2601 to prevent stray light from the surroundings accessing the glass aperture 2102. The curtain can be made from any visually opaque material, with fabric and polymer films the most preferred materials.

During skin imaging it is preferable to have the skin flat and pressed against the glass aperture of the handset such that an even illumination is provided across the skin surface. Consequently, a degree of force is required when pressing the handset onto the skin surface. An operator familiar with the device will have experience of the amount of pressure required, but an unfamiliar operator may provide too much or too little force. Applying too much force is detrimental in situations where the apparatus to which the invention pertains is used for mapping the topology of the dermal-epidermal junction.

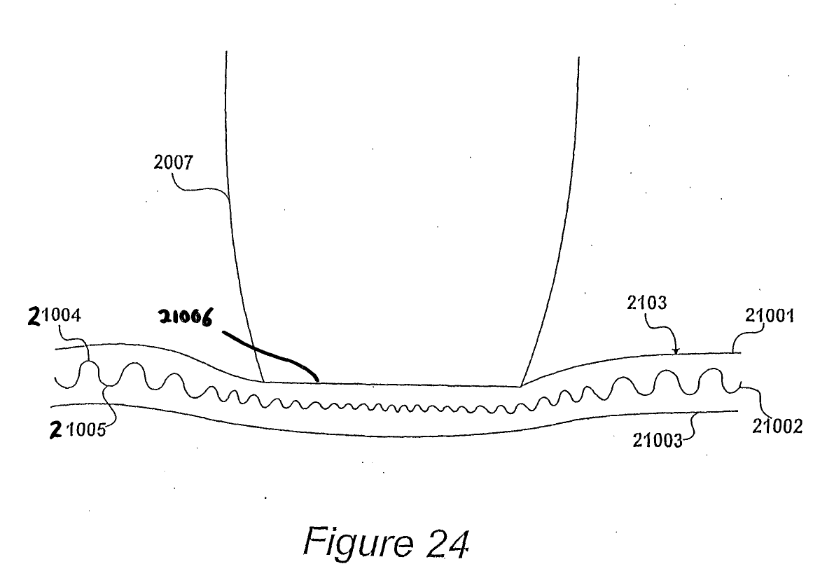

FIG. 24 illustrates a schematic representation of a nose cone 2007 of the skin illumination apparatus in contact with a skin surface 2103. The skin is shown in cross section illustrating the stratum corneum 21001 dermo-epidermal junction 21002 and the boundary between the dermis and the sub-cutaneous tissue 21003. In normal skin the dermo-epidermal junction exists as an undulating layer of peaks and troughs which define finger like projections or “papillae”. In FIG. 24 this layer is shown schematically as peaks 21004 and troughs 21005. If the nose cone 2007 is pressed against the skin surface 2103 with more force than is required the skin surface is compressed between the nose cone 2007 and the pressure exerted by the underlying sub-cutaneous tissue 21003. Hence, the thickness of the skin is reduced and the dermal papillae are squashed as illustrated at 21006. This may lead to false interpretation of the data, particularly in conditions where a flattening of the dermal papillae is diagnostic feature of a skin condition such as, for example, basal cell carcinoma.

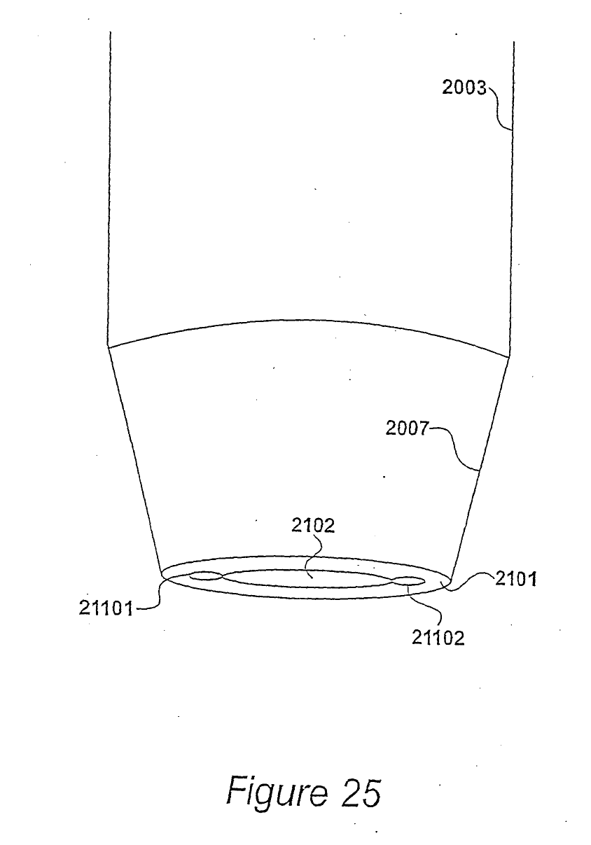

FIG. 25 illustrates a modified nose cone receivable on the handset of the apparatus to which the invention pertains. The nose cone 2007 comprises the usual nose cone end 2101 which defines a transparent glass aperture 2102 through which the skin is illuminated. In addition, mounted within the nose cone end 2101 are two load cells 21101 and 21102 which are contacted with the skin. The load cells produces an electrical output corresponding to the pressure. The load cell is calibrated to detect an acceptable range of pressure between the skin surface and the nose cone 2007. If the pressure exceeds a predetermined threshold level, the apparatus is configured to prevent an image been obtained and thus prevent a false representation of the skin surface being imaged.

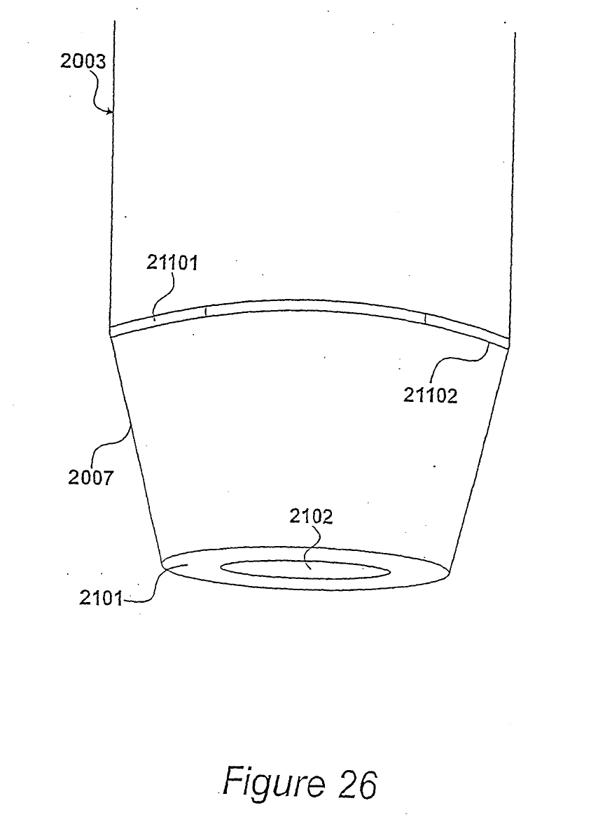

FIG. 26 shows an alternative embodiment of the present invention whereby the nose cone 2007 is provided with a load cells 21101 and 21102 situated between the nose cone and handset 2003. Applying pressure to the nose cone 2007 in turn transfers pressure to the junction between the nose cone 2007 and the handset 2003. Similarly, to the embodiment illustrated in FIG. 25, the load calls are configured to detect pressures above a predetermined maximum.

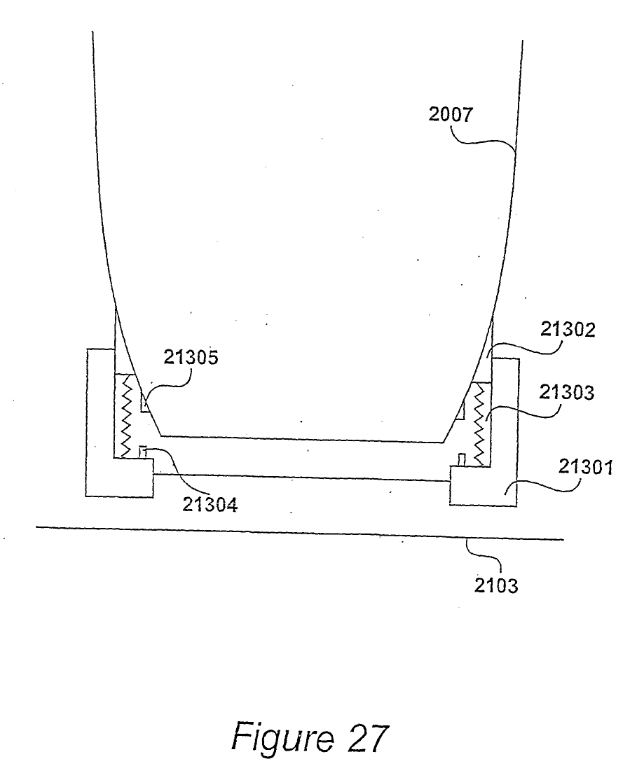

A mechanical means by which a maximum threshold pressure is detected is shown in FIG. 27. The nose cone 2007 is provided with a circular skin-contacting member 21301 attached to a support 21302 on the nose cone by a resilient spring 21303. A transparent film 2302 is mounted within the aperture defined by the skin-contacting member 21301. The nose cone is orientated over an area of skin to be imaged and pressed against the surface 2103. The skin contact member is forced towards the nose cone body 2007, compressing the resilient spring 21303. If the pressure exceeds a defined threshold, the skin contact member is forced such that the protuberances 21304 contact the microswitch 21305 mounted on the nose cone 2007. The actuation of the microswitch triggers an alarm or visual message alerting the operator to the over pressuring of the skin area and prevents an image being obtained until the pressure is reduced below the predetermined threshold value.

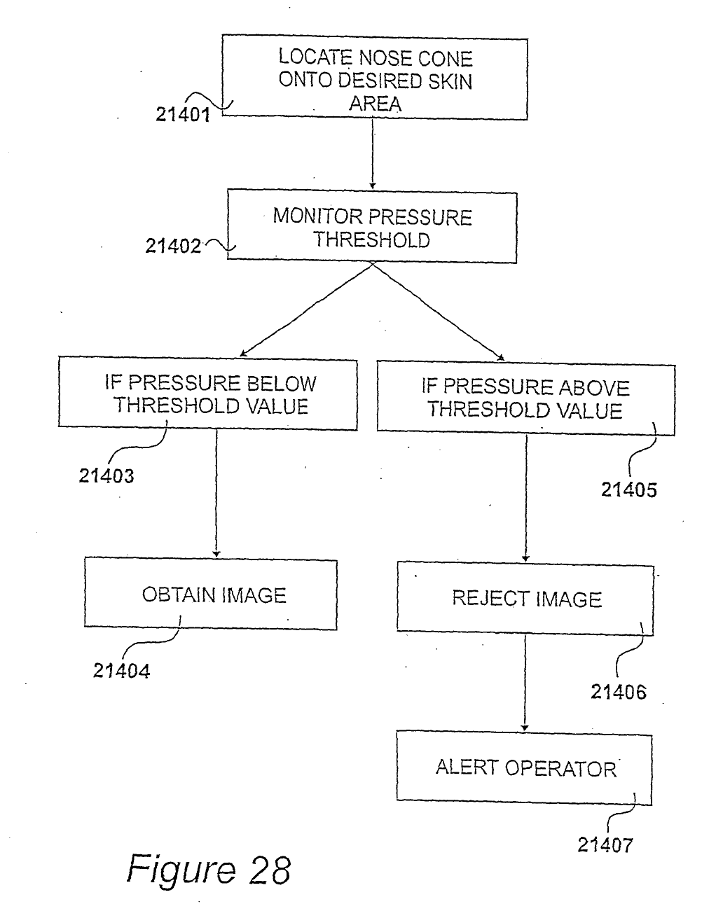

FIG. 28 is a flow chart illustrating an example of an operational sequence employed to prevent over pressurising an area of skin. The nose cone is located over the desired area of skin to be imaged 21401. The load threshold is monitored by the pressure detection means 21402. If the pressure is too high 21405, indicated by a signal from the pressure detection means, then the image is rejected 21404 and the operator alerted by audio alarm of visual signal 21407. If the pressure is below the defined threshold 21404, the image is obtained as per the normal operational procedure of the skin measurement apparatus 21407. Consequently, when the operator is alerted to the over pressuring of the skin, the pressure applied may be reduced to below the threshold upon which an image is obtained as per the standard imaging procedure.

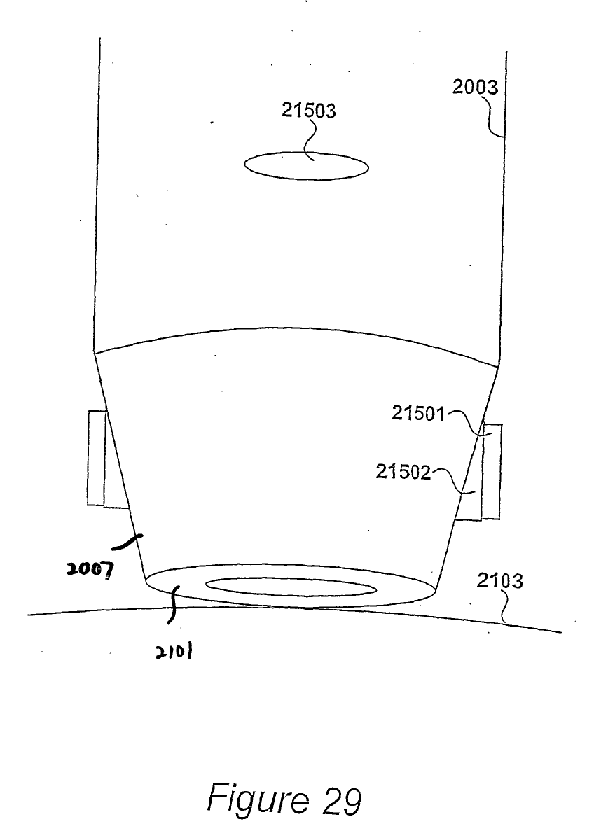

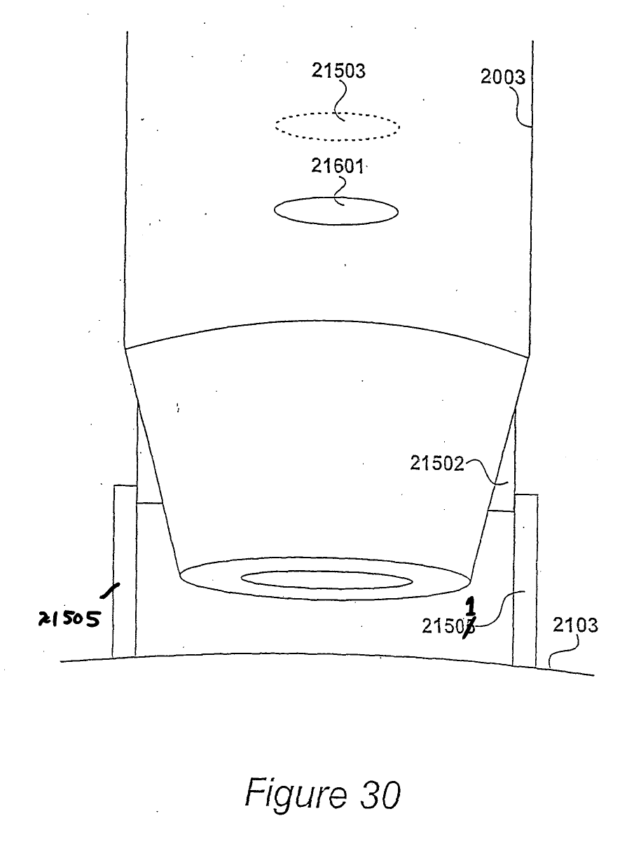

A further modification to the nose cone is illustrated in FIG. 29. During use of the apparatus to which the invention relates, it is advantageous to provide a “clinical view” which, in other words, is an image of the skin surface. In FIG. 29, the nose cone 2007 of the handset is equipped with a two leg members 21501a and 21505b rotatably mounted onto a support 21502. In FIG. 29, the legs are in the “up position” and the handset is configured for illuminating the skin surface and detecting the light remit ted. A lens 21503, mounted within a handset 2003, focuses the remitted light onto a detector (not shown). To obtain a clinical view the legs are rotated into a down position, as illustrated in FIG. 30. The nose cone end 2101 is lifted from the skin surface 2103 and the position of the lens adjusted to focus on the skin surface 2103. In a preferred embodiment the detector is provided with an auto-focus system which automatically moves the lens 21503 mounted within the handset to focus on the skin surface 2103. Alternatively, the lens may be moved to predetermined position by a mechanical means associated with the rotatable leg members. For example, when the leg members are in a “up position” the lens resides in a fixed position 21503 during imaging of chromophores within the skin and upon moving the leg members to a ‘down position’ the lens is moved to a second predetermined position 21601 to focus on the skin surface.

Although the nose cone is spaced above the skin surface by the leg members in the embodiment illustrated in FIGS. 29 and 30, any spacing means would suffice, such as, for example, a spacer ring or foam ring of defined dimensions such that the nose cone is spaced an optimal distance from the skin surface.

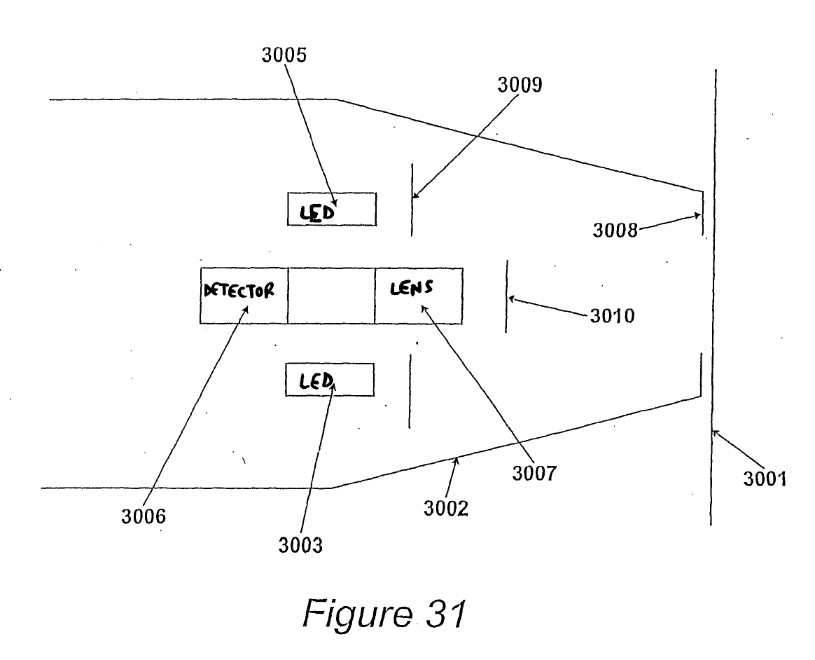

FIG. 31 relates to a portable device of assessing the presence of melanin within the dermis of skin.

Knowledge of the presence or absence of melanin in the dermis of the skin has been shown to be particularly useful in the assessment of skin conditions such as pigmented lesions. One example of a pigmented lesion is melanoma, the most serious form of skin cancer, the pigment melanin can be present in the papillary dermis because malignant melanocytes have crossed the boundary between the papillary dermis and epidermis. Therefore the presence of dermal melanin is well known to be associated with melanoma although it is also associated with a number of benign conditions.

A non invasive optical technique for detecting the presence of dermal melanin has been described in GB 9624003.1, GB 002124 and GB 0016690.0. These patents have been embodied in a large device called a SIAscope which has a high build price and is generally clinic based. The SIAscope measures over an area returning information on a multitude of features including the total amount of melanin, collagen, blood and dermal melanin. An example of a SIAscope is illustrated in FIGS. 13 and 14.

There is a need, however, for a low cost portable device allowing fast assessment of individual features as has already been discussed in relation to FIGS. 1 to 12.

According to the present invention there is a self-contained portable device to detect the presence of dermal melanin in the skin. To be low cost this invention returns a numerical response, which represents the amount or depth of dermal melanin. This response could be displayed as, but is not restricted to, a number, graphically or in a binary fashion. To achieve low cost the device images a small area of skin and therefore requires the user to move the device over the skin. To allow this form of operation it is desirable for the device to produce a very fast, apparently real time, response. There are a number of reasons that a device of this type may have a limited life, including, but not limited to, calibration drift, environmental effects, degradation of the battery, effects of cleaning materials. To achieve this controlled lifetime the device may include, but not limited to, timers to measure usage or counters to measure number of uses or battery charge cycles.

A specific embodiment of the invention will now be described by way of an example with reference to FIG. 31.

The device consists of a series of light sources 3003 and 3005 in this case four sources are arranged around the central axis of the device, each source has specific spectral characteristics, each source is arranged to project light onto the surface of the skin 3001 to be analysed. These light sources may each consist of a single Light Emitting Diode, LED, an array of similar LEDs, or broad spectrum light source together with band pass filters. In the case of broad spectrum sources these may be, for example, xenon flash tubes or incandescent bulbs.

The light incident on the skin is measured by an electronic detector 3006. The light incident on an optional surface of known reflectance 3008 may also be measured for calibration. This detector may be a single photo detector or an array of photo detectors for example a charge coupled array device or Complimentary Metal Oxide Semiconductor, CMOS array.

The device may be used with different areas on the array identified as active areas for each measurement of light incident oh the skin or light reflected from the skin. A lens 3007 is used to focus an image of the skin onto the detector 3006. The device may also be used to analyse a single area or point of skin.

It is advantageous to exclude ambient light to increase the accuracy of these measurements. The housing 3002 is designed for this purpose.

It is advantageous, but not essential to polarise the light from the light sources using a linear polarising filter 3009, and to polarise the light entering the light detectors in a similar manner 3010, such that both these filters have axes of polarisation at 90 degrees to one another. This ensures that specular reflections from the skin surface do no effect measurements of light scattered from within the skin.

The signal from the light-measuring device is connected to a processing means. This processing means also controls the light sources 3003 and 3005 such that measurements of light intensity both incident on the skin and scattered from it can be made in sequence, energising and measuring results for each light source in turn.

The processing means may also obtain a measurement of light with no light sources enabled to allow calibration of the detector, and to check that ambient light is adequately excluded.

The procedure for calibration is such a device is described fully in GB 002124. These calibration steps may be required, but they are not essential.

The processing means is therefore able to calculate the percentage reflectance of the skin within the spectral range of each light source, by calculating the ratio of reflected to incident light.

This data is stored temporarily by the processing means. The processing means uses the method described in GB 9624003.1 and GB 0002124 to determine the presence of dermal melanin.

If this method concludes dermal melanin is present the processing means will enable an indicator, which can take the form of a light, audible warning or message on an integral message display to be operated.

The device is particularly useful when miniaturised and operated by battery.

The processing means may include non-volatile electronic memory in which is stored data on the sensitivity and linearity of the light detector, 3006, at each spectral band used by the light sources. This information is used by the processing means to calibrate the signal from the light detectors for variations in light detector performance.

The device may include a counter such that after a pre set number of operations the device will become inactive. This ensures that the user returns the device to the manufacturer so that the calibration of the light sources and detectors can be checked.

The device is used by placing it on the skin in such a way that ambient light is excluded. The device is energised by pressing a button that activates the processing means so that the readings are taken and calculations performed as described above.

The operator can then use information on the presence of dermal melanin, together with other clinical information, for the diagnosis of melanoma.

In an alternative embodiment it is envisaged that this calibration area, 3008 would not be necessary.

In an alternative embodiment the lens, 3007 may be replaced by a simple aperture or may be omitted due to the detector design.

In an alternative embodiment the device lifetime may be determined by a combination of timers and or counters to determine the end of calibration life of the device.

The device of FIG. 31 may be designed to be hand-held and in the form of a “pen” as with the embodiment shown in FIG. 1.

1. A hand-held device for the determination of the concentration and distribution of chromophores within an epithelial surface, comprising
illumination means configured to illuminate an area of said epithelial surface
detection means to convert the intensity of remitted light into an electrical signal
processing means configured to analyse the difference in intensity of one or more of said chromophores across a lesion; and
display means for displaying an output from said processing means.

2. A method of interpreting the distribution of chromophores within a lesion of an epithelial tissue, said method comprising the steps of:
illuminating the epithelial surface with a wavelength of light corresponding to a chromophore
detecting the intensity of light remitted from the skin surface to form a data set
determining the variation in intensity of said remitted light across said lesion, and
providing an output corresponding to the significance of the variation in the concentration of said chromophore across the lesion.

3. A method of interpreting the distribution of chromophores within a lesion of an epithelial tissue, said method comprising the steps of:
illuminating an epithelial surface with a wavelength of light corresponding to a chromophore
determining the intensity of light remitted from said lesion to form a first data set
illuminating an epithelial surface with a wavelength of light corresponding to a second chromophore
obtaining an image of light remitted from said epithelial tissue to form a second data set; wherein variations in said first and second data sets are determined and an output corresponding to the degree of variation within each data set generated.

4. A skin illumination and remitted light detection apparatus, comprising
a light tube defining a transparent glass aperture contactable with the skin,
illumination means configured to transmit light to said light tube,
detection means to detect light remitted from the skin,
wavelength selection means to select the wavelength of light incident on said detection means,
illumination intensity selection means to select the intensity of light incident on the detection means, characterised in that
said apparatus further comprises barrier means configured to prevent direct contact between said glass aperture and said skin.

5. A skin illumination and remitted light detection apparatus, comprising
a light tube defining a transparent glass aperture contactable with the skin,
illumination means configured to transmit light to said light tube,
detection means to detect light remitted from the skin,
wavelength selection means to select the wavelength of light incident on said detection means,
illumination intensity selection means to select the intensity of light incident on the detection means, characterised in that
said apparatus further comprises an ambient light exclusion means to prevent ambient light accessing said glass aperture.

6. A skin illumination and remitted light detection apparatus, comprising
a light tube defining a transparent glass aperture contactable with the skin,
illumination means configured to transmit light to said light tube,
detection means to detect light remitted from the skin,
wavelength selection means to select the wavelength of light incident on said detection means,
illumination intensity selection means to select the intensity of light incident on the detection means, characterised in that
said apparatus further comprises a pressure detection means configured detect a threshold level of pressure between said light tube and the skin.

7. A skin illumination and remitted light detection apparatus, comprising
a light tube defining a transparent glass aperture contactable with the skin,
illumination means configured to transmit light to said light tube,
detection means to detect light remitted from the skin,
wavelength selection means to select the wavelength of light incident on said detection means,
illumination intensity selection means to select the intensity of light incident on the detection means, characterised in that
said apparatus further comprises a means for locating said glass aperture a defined distance from said skin surface such that a clinical view of the skin surface is obtained.

8. A device as claimed in claim 1 incorporating means to indicate to a user that the useful life of the device is at an end.

9. A device as claimed in claim 8 in which the said means comprises a timer and/or counter which records the total time the device has been in use or the number of times the device has been used respectively.
